(19) United States
(12) Patent Application Publication
(10) Pub. No.: US 2007/0209938 A1
(43) Pub. Date: Sep. 13, 2007

(54) Method and apparatus for biopolymer analysis

(76) Inventors: Jianzhong Zhang, Brea, CA (US); Yu Fang, Brea, CA (US)

Correspondence Address: MR. JIANZHONG ZHANG, 1823 MOORPARK DR, BREA, CA, 92821, US

(21) Appl. No.: 11/373,602

(22) Filed: Mar. 13, 2006

(51) Int. Cl. C07K 1/26

(52) U.S. Cl. 204/451

(57) ABSTRACT

Method that facilitates sensitive biopolymer characterization is disclosed. Post-column fluorescence detection is uniquely suitable for dynamically labeled biopolymers such as protein. Detection sensitivity has been enhanced by adding fluorescent stain and organic modifier to non-gel sieving matrix and by increasing the viscosity of sheath fluid. A detection limit better than silver staining is possible using the method according to the present invention. Throughput may be increased by parallel operation of capillary array or an array of sheath flow cuvettes.

### FIELD OF THE INVENTION

The present invention relates generally to method and apparatus for biochemical analysis. Particularly, the present invention provides method and apparatus for separation and detection of biopolymers such as protein.

### BACKGROUND AND SUMMARY OF THE INVENTION

Biological systems are very complex in nature. Biopolymers such as proteins exist in life process in a wide variety of forms, differing in size, charge and chemical composition; proteins also exist in a wide range of concentrations, ranging from mg/ml to pg/ml or even lower. Accordingly, analytical systems used to characterize proteins need to have high resolution and high sensitivity. In addition, parallel operation of a number of biological samples is often necessary, requiring an analytical system to provide increased throughput.

Traditional protein size characterization is usually conducted with gel electrophoresis, in which a layer of gel is sandwiched between two glass plates. The gel is composed of a porous gel structure, a buffer agent and a negatively-charged detergent (normally sodium dodecyl sulfate, or SDS). The concentration of the detergent is in the range of 0.1% (w/v) so that the detergent and sample protein form a dynamic SDS-protein complex, resulting in a constant charge-to-size ratio to the protein molecules. An electric field is applied across the gel matrix. Protein molecules migrate across the gel matrix under the influence of the electric field with the migration distance being inversely related to the size of the protein molecules.

The gel thus generated may be stained with either Coomassie Brilliant Blue or Silver to reveal the protein profile in the form of visible bands. Coomassie Brilliant Blue gives wide detection range but Silver has the sensitivity of detecting as little as a few nanograms of proteins. Newer addition to the protein detection is a number of fluorescent stains, such as NanoOrange, Sypro Orange and Sypro Red, which enable Silver stain sensitivity and are more convenient to use (Steinberg, T. H. et al., “Sypro Orange and Sypro Red Protein Gel Stains: One-Step Fluorescent Staining of Denaturing Gels for Detection of Nanogram Levels of Protein”, Anal. Biochem., 239: 223-237, 1996). In reality, the generation of a stained gel is rather time-consuming and labor-intensive.

An alternative approach to the traditional gel electrophoresis is capillary gel electrophoresis. In capillary gel electrophoresis, a fused silica capillary tube of internal dimension of about 50 μm is filled with a non-gel sieving matrix, which is composed of an entangled polymer network (instead of a porous gel used in conventional gel electrophoresis), a buffer agent and a negatively charged detergent. Capillary gel electrophoresis has inherited size-based separation mechanism from the traditional gel electrophoresis. Capillary gel electrophoresis outperforms its traditional counterpart in terms of separation speed and separation efficiency. Furthermore, modern capillary electrophoresis consumes very little sample and provides automation. The drawback, however, is the lack of detection sensitivity, which has limited its application, at least in the case of protein characterization.

At present, on-column absorbance and on-column fluorescence are among the most widely used capillary gel electrophoresis detection methods for protein detection, providing assay sensitivity comparable to that of Coomassie Brilliant Blue and Silver, respectively. Unfortunately, most proteins do not fluoresce in the visible wavelength; additional chemical reaction is required to label proteins with certain fluorophores. This reaction inevitably introduces a multiple-labeling problem, which in most cases will change the characteristics of the proteins and smear the subsequent separation.

A few attempts have been made by capillary electrophoresis community to adopt the fluorescent stains and incorporate a dynamic labeling strategy to protein detection. The dynamic labeling method eliminates pre-labeling reaction and multiple-labeling problem. However, Lin, L. et al. (“Dynamic Labeling during Capillary or Microchip Electrophoresis for Laser-Induced Fluorescence Detection of Protein—SDS Complexes without Pre- or Postcolumn Labeling”, Anal. Chem., 73: 4994-4999, 2001) show that the stain remaining in the separation matrix interacts with SDS micelle, resulting in a high background, which diminishes overall detection sensitivity. Giordano, B. C. et al. (“Microchip Laser-Induced Fluorescence Detection of Proteins at Submicrogram per Milliliter Levels Mediated by Dynamic Labeling under Pseudonative Conditions”, Anal. Chem., 74: 4705-4714, 2004) and Harvey, M. D. et al. (“Subnanomolar Detection Limit for Sodium dodecyl sulfate—Capillary Gel Electrophoresis Using A fluorogenic Noncovalent Dye”, Electrophoresis, 19: 2169-2174, 1998) report that high sensitivity can be achieved through excessive SDS dilution. Unfortunately low SDS concentration leads to poor separation as showed in the above-cited references. In fact, the separation efficiency from these attempts is not better than that of traditional gel electrophoresis.

The inventors have therefore proposed an analytical method, which provides high detection sensitivity without sacrificing separation efficiency.

In one aspect of the present invention, there is provided a method, comprising providing analytical separation of biopolymers using a capillary tube with a separation matrix disposed within.

In a further aspect of the invention, the said separation matrix comprises a detergent-containing non-gel sieving matrix, a fluorescent stain, and at least an organic modifier or a possible combination of different organic modifiers. The concentration of the said detergent in the said separation matrix is lower than 0.1% (w/v) after addition of the said organic modifier or modifiers; while the concentration of the said organic modifier or modifiers are in a range for both enhanced detection sensitivity and enhanced separation efficiency.

In a still further aspect of the invention, sheath flow cuvette is adapted for post-column fluorescence detection of dynamically labeled biopolymers. The sheath fluid comprises at least a hydrophilic polymer or a possible combination of hydrophilic polymers, which makes the sheath fluid more viscous.

In one embodiment of the invention, there is provided a sheath flow cuvette array apparatus for parallel capillary electrophoresis.

Further aspects and features of the present invention will now be described with regards to the accompanying drawings, which assist in illustrating various features of the invention. However, it should be appreciated that the drawings do not constitute limitations to the scope of the present invention.

### BRIEF DESCRIPTION OF THE DRAWINGS

FIG. 1 is a schematic view of an exemplary single sheath flow cuvette apparatus according to the invention.

FIG. 2 is a sectional view of the sheath flow cuvette assembly between line 1-1 and line 2-2 of FIG. 1.

FIG. 3 is a sectional view of the sheath flow cuvette 12 between line 3-3 and line 2-2 of FIG. 1.

FIG. 4 is a cross-sectional view along line 3-3 in FIG. 1.

FIG. 5 is a sectional view between line 3-3 and line 2-2 of FIG. 1, along with the elements required for excitation of laser-induced fluorescence.

FIG. 6 is a cross-sectional view along line 2-2 of FIG. 1 and the elements required for both excitation and detection of laser-induced fluorescence.

FIG. 7 is a schematic view of an exemplary sheath flow cuvette array apparatus according to the invention.

FIG. 8 is a sectional view between line 7-7 and line 8-8 of FIG. 7.

FIG. 9 is a cross-sectional view along line 7-7 of FIG. 7.

FIG. 10 is a sectional view between line 7-7 and line 8-8 of FIG. 7 and the elements required for excitation of laser-induced fluorescence.

FIG. 11 is a cross-sectional view along line 8-8 of FIG. 7 and the elements required for both excitation and detection of laser-induced fluorescence.

FIG. 12 is a graph showing the data generated from apparatus of FIG. 1 and methods according to the invention.

### DETAILED DESCRIPTION OF THE INVENTION

With on-column fluorescence detection, light scattering at curved capillary interface presents a technique challenge to detection sensitivity. On the contrary, post-column detection usually performs better in terms of detection sensitivity. Dovichi, N. J. et al. disclosed such a detection scheme based on a sheath flow cuvette (“Laser-induced fluorescence of flowing sample as an approach to single-molecule detection in liquids”, Anal. Chem., 56: 348-354, 1984). The present invention discloses another use of sheath flow cuvette as a post-column fluorescence detector in detecting dynamically labeled proteins. In another embodiment of the present invention, an array of the said sheath flow cuvettes is arranged for parallel operation.

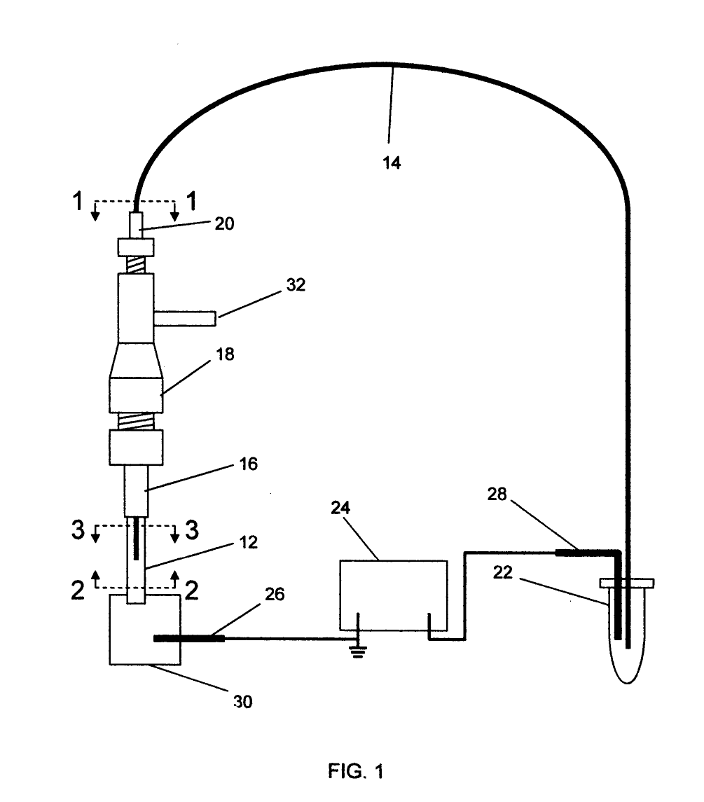

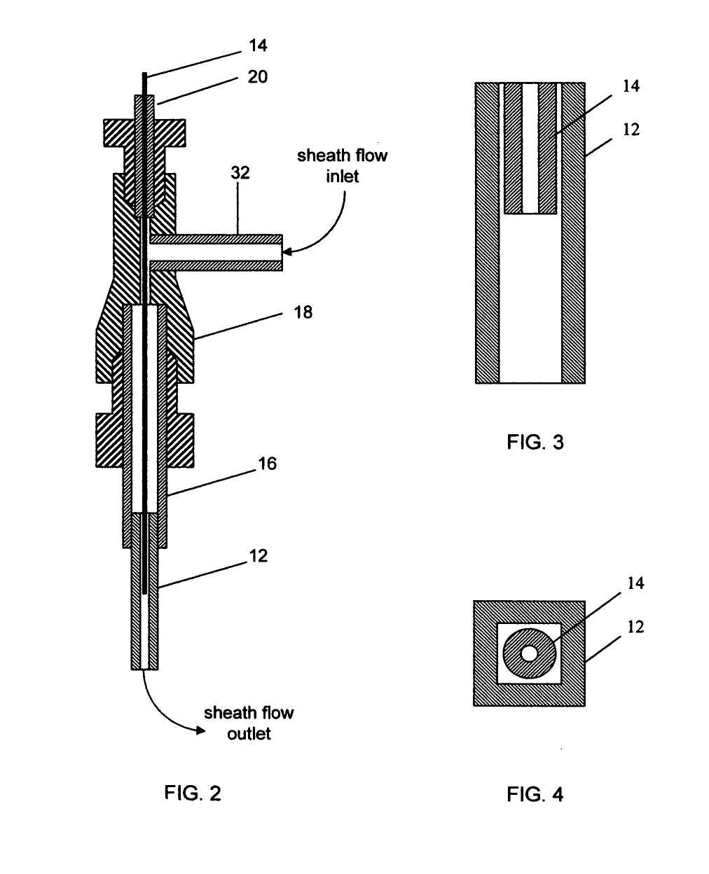

Referring to FIG. 1, the method comprises using a sheath flow cuvette 12 and a capillary tube 14. The first end of the capillary tube 14 terminates in the interior chamber of the sheath flow cuvette 12. The second end of the capillary tube 14 extends into a well 22. The capillary tube 14 is held in a modified MicroTight adapter 18 and a sleeve 20. The capillary tube 14 is a conventional fused silica capillary, about 50 μm ID and 375 μm OD. The sheath flow cuvette 12 is a borosilicate or fused silica chamber, glued inside a stainless steel tube 16 that is fixed with the MicroTight adapter 18. The chamber has an interior dimension of 400 μm×400 μm, allowing the capillary tube 14 fit snugly inside. The sheath flow cuvette assembly, the inlet and outlet of the sheath flow are shown in FIG. 2, while the sheath flow cuvette is shown in FIG. 3 and FIG. 4 in more detail. The sheath flow inlet 32 is on the modified MicroTight adapter 18 while the outlet is at the bottom of the sheath flow cuvette 12. A high voltage power supply 24 is connected to a first electrode 26, which is sealed in a waste container 30. The waste container is placed downstream the sheath flow cuvettes 12. The high voltage power supply 24 is also connected to a second electrode 28, which is dipped in the well 22. When the capillary tube 14, sheath flow cuvette 12, the waste container 30 and the well 22 are filled with conducting material, a high voltage may be applied across the capillary tube 14 using the high voltage power supply 24 to drive sample proteins from the second end to the first end of the capillary tube 14. The ground first electrode 26, the waste container 30, the sheath flow cuvette 12, the capillary tube 14, the well 22, the second electrode 28 and the high voltage source 24 form a complete circuit. Because capillary ID is much smaller than the chamber dimension, most of the voltage will be on capillary tube 14 and the voltage on sheath flow cuvette 12 can be neglected.

The capillary tube 14 is in contact with well 22 and is filled with a separation matrix. The filling and re-filling may be achieved through the application of a pressure to the well 22 when it contains the separation matrix. At different stages of the separation operation, the well 22 may contain different material such as a protein sample or a detergent-containing run buffer, which may be identical to the separation matrix.

For clarity, non-gel sieving matrix refers to a solution of an entangled polymer, a buffer agent and a detergent; whereas the separation matrix disclosed in the present invention has both organic modifier and fluorescent stain pre-mixed with the non-gel sieving matrix. In the current invention, the addition of up to 30%, preferably from 10 to 15%, organic modifier to the non-gel sieving matrix of original 0.1% (w/v) SDS serves at least dual purposes. First, it is known that the addition of aliphatic alcohol, preferably ethylene glycol, improves the separation efficiency (Bean, S. R. and Lookhart, G. L., “Sodium Dodecyl Sulfate Capillary Electrophoresis of Wheat Proteins. 1. Uncoated Capillaries”, J. Agric. Food Chem., 47: 4246-4255, 1999). More importantly, the addition of the organic modifier has moderately lowered the detergent concentration to a sub-micellar range where SDS is still sufficient to form SDS-protein complex but not enough to form SDS micelle. In other words, the addition of the organic modifier has created an environment for maximized fluorescence signal relative to the background noise. This high signal-to-noise ratio translates into high fluorescence detection sensitivity.

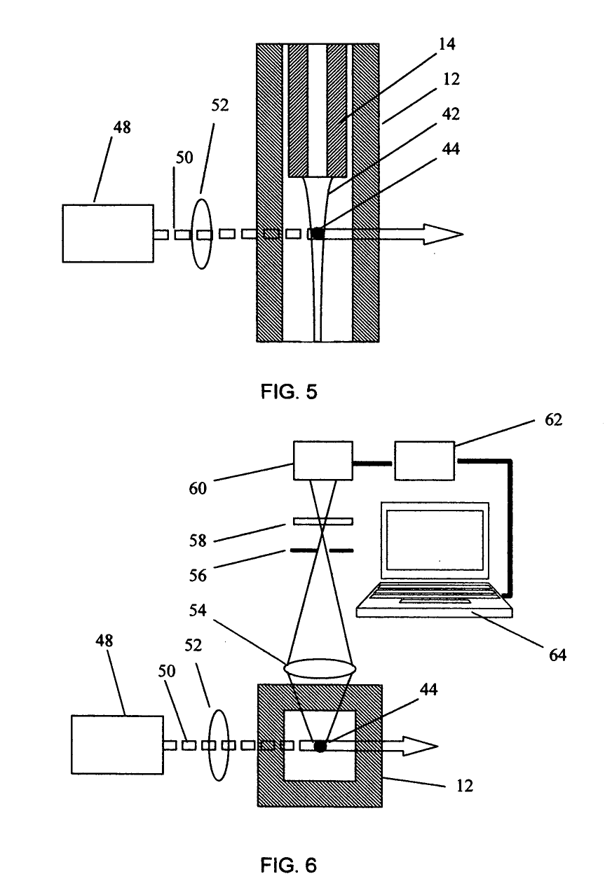

Referring to FIG. 5, there is shown a section of the sheath flow cuvette of FIG. 1 in its operation mode. It is illustrative to add sample stream 42, laser beam 50, and laser-induced fluorescence 44. To facilitate fluorescent detection, the first end of the capillary tube 14 terminates in the center of the sheath flow cuvette 12 having one or more optical windows. The beam 50 from a laser 48 or a collimated light source is shaped with a suitable focusing lens 52 and is perpendicularly positioned to intersect the sample stream 42 one hundred micrometers or more downstream the first end of the capillary tube 14. The sheath fluid may be driven by pressure, or by gravity, to form a continuous, pulse-free laminar flow, surrounding the sample stream 42 that passes through the optical windows of the sheath flow cuvette 12, where the fluorescence 44 from the passing sample stream 42 is excited. A linear speed in the order of 1 mm/s downstream the first end of the capillary tube may be adequate for the sheath flow.

A laser line is chosen to match the absorbance maximum of the fluorescent stain. For example, the 488-nm line from an argon ion laser can be used to excite Sypro Orange, while the 543-nm line from a He—Ne laser, or the 532-nm line from a frequency-doubled YAG laser, can be used to excite Sypro Red.

The supplied sheath fluid also needs to be grafted to suit post-column fluorescence detection of dynamically labeled proteins. Addition of at least a hydrophilic polymer, such as cellulose derivatives, polyethylene glycol, polyethylene oxide, dextran, and modification and mixture thereof, to a sheath fluid has proved very effective, partly because of the increase in viscosity of the sheath fluid. Higher viscosity makes the SDS micelle less likely to form, thus reducing the background fluorescence. Higher viscosity of the sheath fluid also suppresses the eddy effect around the corner of the capillary end, stabilizing the sheath flow at the fluorescence detection window. The concentration of the polymer in the sheath fluid depends on a number of factors such as the molecular weight of the polymer, the chamber dimension of the sheath flow cuvette and operation temperature. Again a linear speed in the order of 1 mm/s downstream the first end of the capillary tube may be maintained for the sheath flow.

It should be noted that driving neutral species such as hydrophilic polymers along with the sheath fluid needs the application of pressure, gravity or the like, but not electric field. Pressure or gravity is more universal in driving different species, charged or neutral, along with the sheath fluid, whereas electric field is only limited to charged species.

Referring to FIG. 6, there is shown a cross-section of the sheath flow cuvette 12, laser-induced fluorescence 44 and the elements required for laser-induced fluorescence excitation and detection. A collection lens 54, which can be a microscope objective or a camera lens, is positioned at right angle to the laser beam 50 to image fluorescence 44 from the sample stream onto a photon detector 60, such as a photomultiplier tube (PMT), an avalanche photodiode or a charge-coupled device (CCD). A spatial filter 56 and a spectral filter 58 are placed in front of the photon detector 60. The filters allow transmission of fluorescence and rejection of stray light and scattering. The photon detector output, which should be proportional to the intensity of the fluorescence or the quantity of the sample protein, is passed through a data acquisition unit 62 to a computer 64 for data acquisition and recording.

The method of post-column detection of dynamically labeled protein disclosed in the present invention can be readily applied to multiple capillary systems that have been patented in the past.

An example of such multiple capillary systems is what Dovichi and I have disclosed in U.S. Pat. No. 5,439,578 issued Aug. 8, 1995. That patent discloses a detection of sample streams from multiple capillary tubes terminated in a rectangle sheath flow cuvette. Sheath fluid entrains individual sample streams from these capillaries and a laser beam is positioned across the end of the capillaries. Light emitted from these sample streams is simultaneously detected with a fluorescence detection system.

In one embodiment of the analyzer that disclosed in the above-identified patent, the inner walls of the cuvette are slanting, making the chamber slightly bigger at the top than at the bottom. This configuration, even though very compact and good at aligning the capillary ends, puts the capillary OD dimension on a very tight tolerance, which is not readily available from commercial sources. Besides, the fluorescence detection relies on an array of discrete components such as photodiodes, optical fibers and gradient-index lenses, which are difficult to assemble and align.

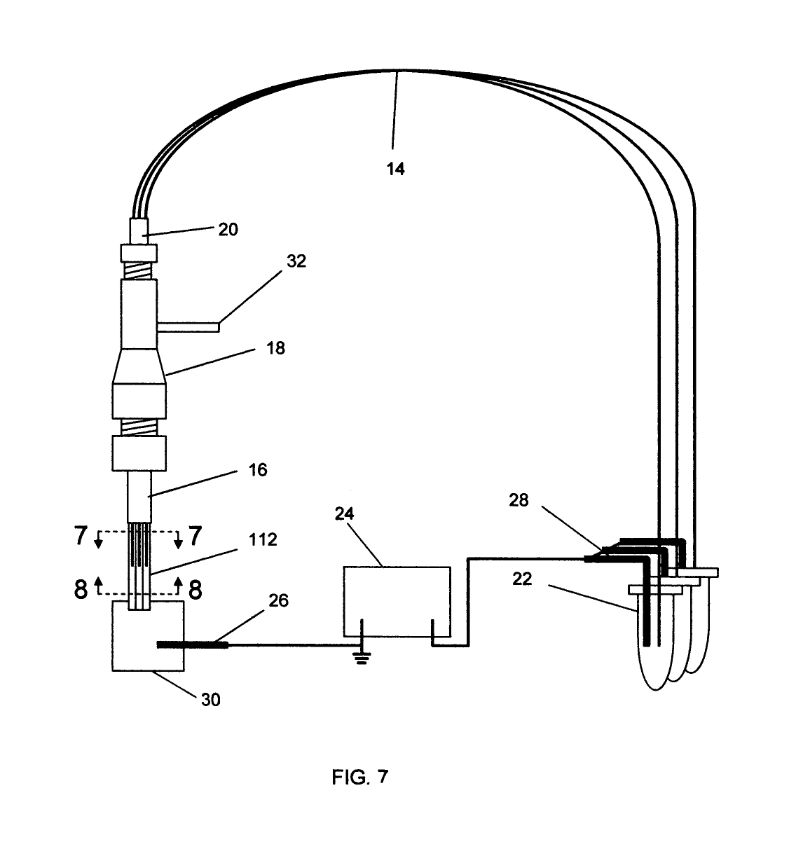

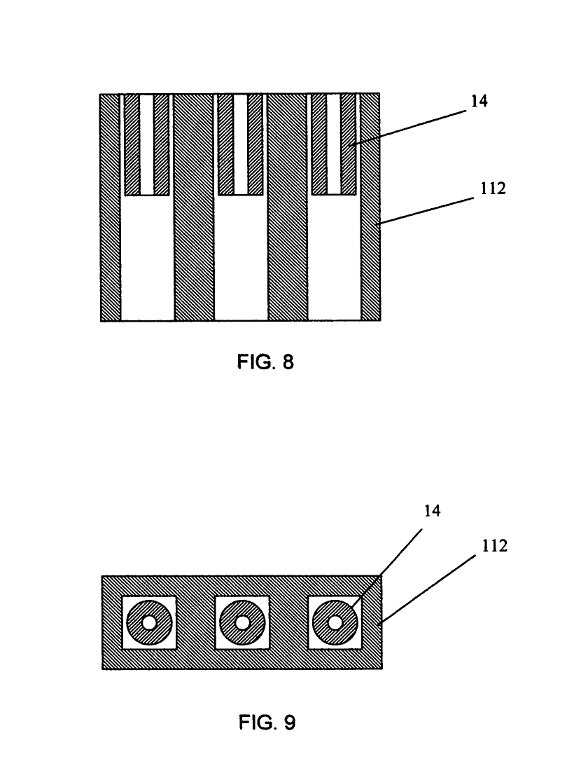

Instead of the capillary array, the inventors disclose an embodiment of a sheath flow cuvette array, shown in FIG. 7, for parallel operation of capillary gel electrophoresis. FIG. 8 and FIG. 9 show three identical sets of sheath flow cuvettes in more detail. In this cuvette array 112, the tolerance of capillary OD is no longer an issue. Adjacent sheath flow cuvettes may be glued side-by-side with optical adhesive to reduce the number of air/glass optical interfaces. The cuvette array shares a single sheath fluid inlet 32, a single sheath fluid waste 30 and a single high voltage power supply 24. The second ends of the capillary tubes 14 spread out into different wells 22; each contains a different sample and has its own second electrode 28.

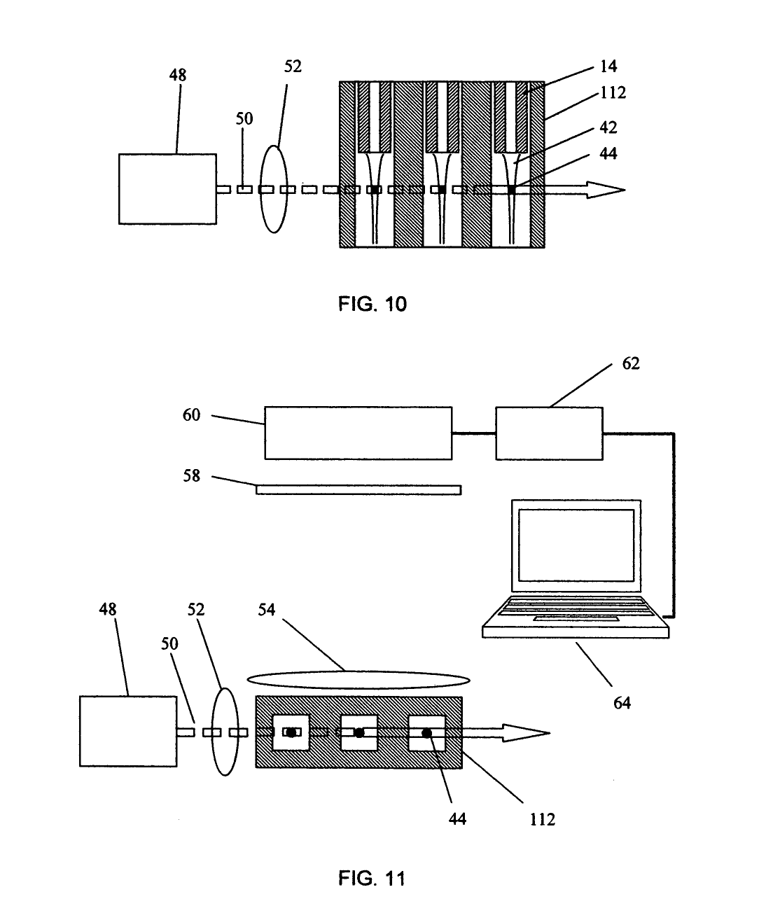

Referring to FIG. 10, a beam 50 from a laser 48 or other collimated light source is required for excitation of fluorescence 44 from the sample streams 42. The beam 50, which is positioned 100 μm downstream the first end of the capillary tubes, enters the cuvette array from one narrow window and exits from the opposite window. Sample streams 42 are independent and evenly spaced. In addition, fluorescence signals 44 from the sample streams are free from crosstalk.

Referring to FIG. 11, a collection lens 54 and photon detector 60 are placed in front of one wide window and orthogonal to the laser beam 50. The collection lens 54 is a camera lens and the photon detector 60 is a charge-coupled device (CCD). The camera lens 54 images the fluorescence 44 onto certain pixels of the CCD 60. Spatial filter 56, as shown in FIG. 6, may not be required. The CCD output is passed through a data acquisition unit 62, to a computer 64 for data acquisition and recording. The array size can be increased by reducing the physical dimension of the capillary tube and sheath flow cuvette. However, the array size may be ultimately limited by the physical dimension of the CCD 60 and by the laser power reduction incurred at each liquid/glass interface as the laser beam 50 traverses the cuvette array.

The dynamic labeling and post-column fluorescence detection disclosed in the present invention should find application other than protein separation and characterization. With proper design and modification, other biopolymers and polymers that are not bio related, may be the subjects of the present invention.

A person skilled in the art could make immaterial modification of the present invention described and claimed in this patent document without departing from the essence of the invention.

The invention is further illustrated in the following example.

### EXAMPLE

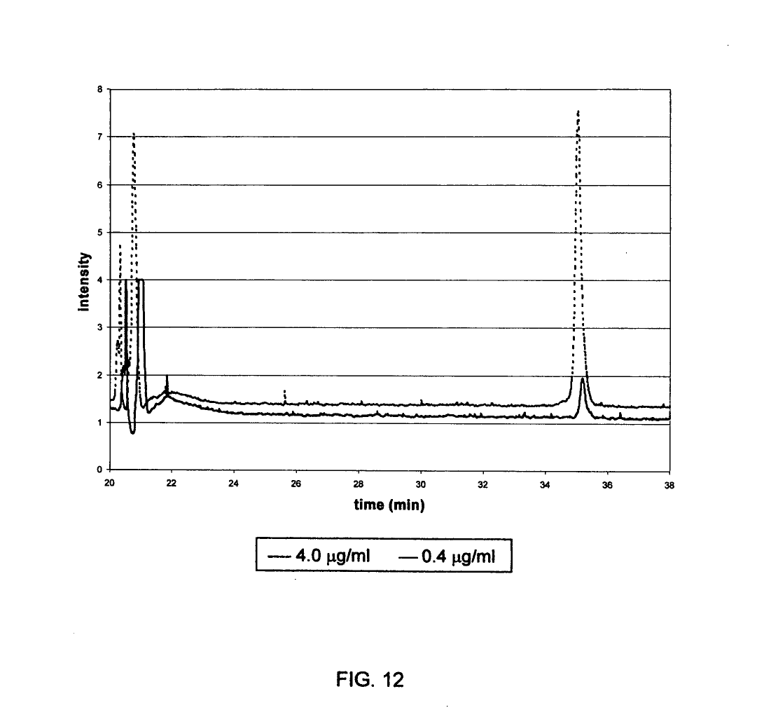

Data has been generated from the apparatus of FIG. 1 using the method described above. The sample is bovine serum albumin, diluted and mixed in 50 mM Tris-HCl buffer containing 0.1% (w/v) SDS. The capillary electrophoresis is driven by a 6 kV voltage over a 25-cm, 50-μm capillary tube filled with a separation matrix containing 15% ethylene glycol and 0.5× Sypro Red. A 5-mW green laser module, operated at 532 nm, is used as excitation source. The laser beam is focused with a 2.5× microscope objective. A spatial filter of 1-mm pinhole and a spectral filter centered at 630 nm are placed in between a 20× microscope objective and a PMT, biased at −800 V. Signal from the PMT is converted with a 330 kΩ/2.2 μF current-to-voltage converter and is digitized at 2 Hz. FIG. 12 shows an electropherogram with a dashed line and a solid line, representing the running of 4.0 μg/ml and 0.4 μg/ml of bovine serum albumin respectively. There is an offset for dashed line. The peaks on the left relate to SDS and SDS micelle while the peaks appearing at 35 minutes relate to bovine serum albumin. Though very preliminary, a plate number >40,000 and an assay detection limit of 0.1 μg/ml, or 1.5 nM, bovine serum albumin have been achieved. This detection sensitivity compares well with that of Silver stain. This detection sensitivity is also very close to capillary fluorescence detection where proteins are pre-labeled with a fluorophore, as reported by Hu, S. et al. (“Analysis of Proteins by Capillary SDS-DALT Electrophoresis with Laser-Induced Fluorescence Detection”, LC•GC Europe, Mar. 2-5, 2002).

The inventors claim:

1. An analytical method for analyzing polymers, preferably biopolymers, comprising:
(a) a capillary tube having a separation matrix disposed within, or
(b) an array of capillary tubes having a separation matrix disposed within,
(c) the said capillary tube/tubes having first and second ends, the first end of the said capillary tube/tubes terminating in a sheath flow cuvette and the second end extending into a biopolymer sample source;
(d) the said sheath flow cuvette having an interior chamber, the first end of the said capillary tube/tubes terminating inside the said chamber,
(e) means to drive the said biopolymers through the said capillary tube/tubes from the second end to the first end of the said capillary tube/tubes,
(f) means to force a sheath fluid into the said chamber to form a laminar sheath flow to draw biopolymer sample from the said capillary tube/tubes into thin sample stream,
(g) a light source to perform fluorescence excitation from the said sample stream, and
(h) a photon detector to perform fluorescence detection of the said sample stream.

2. The said separation matrix of claim 1 comprises a polymer matrix, a buffer agent, a negatively-charged detergent, at least a fluorescence stain, and at least an organic modifier or a combination of different organic modifiers.

3. The said negatively-charged detergent of claim 2 comprises sodium dodecyl sulfate (SDS).

4. The said detergent of claim 2 at the time of separation operation is below its critical micelle concentration, preferably below 0.1% (w/v).

5. The said organic modifier of claim 2 comprises aliphatic alcohols, preferably ethylene glycol.

6. The said organic modifier of claim 2 is at the concentration of up to 30% (v/v), preferably from 10% to 15% (v/v).

7. The said fluorescence stain of claim 2 is Sypro Red, Sypro Orange or the like at the concentration from 0.5× to 3×, preferably from 0.5× to 1×, or at the equivalent concentration.

8. The said sheath fluid of claim 1 comprises at least a hydrophilic polymer or a combination of different hydrophilic polymers, preferably cellulose derivatives, polyethylene glycol, polyethylene oxide, and dextran, and modification and mixture thereof.

9. The said sheath fluid of claim 1 is preferably driven by pressure or gravity.

10. The said sheath flow cuvette of claim 1 has one or more transparent windows at the point of fluorescence detection.

11. The light source of claim 1 is a collimated radiation, preferably from a laser or a laser module, focused 100 μm or more downstream the first end of the said capillary tube/tubes of claim 1 to excite fluorescence from the said sample stream of claim 1.

12. The photon detector of claim 1 is preferably a PMT, an avalanche photodiode or a CCD, positioned at right angle to the said collimated radiation, detecting fluorescence from the said sample stream of claim 1 imaged by a collecting lens, also positioned at right angle to the said collimated radiation and in front of the photon detector.

13. More than one of the said sheath flow cuvettes of claim 1 may be bundled to form a sheath flow cuvette array, mating an array of capillary tubes; each individual cuvette of the said array having the first end of a capillary tube of claim 1 terminating inside while the second end extending into a sample source and each said capillary tube has a separation matrix of claim 1 disposed within.

14. A preferred sheath flow cuvette array in claim 13 is a linear array, having the adjacent said sheath flow cuvettes glued side by side and said capillary tubes of claim 13 terminated at the same level.

15. The said sheath flow cuvette array of claim 13 shares
(a) a high voltage power supply,
(b) a sheath fluid inlet, and
(c) a collimated light source, preferably a laser or a laser module with its collimated radiation shaped and positioned in front of one narrow side of the sheath flow cuvette array of claim 13 and 100 μm or more downstream the first end of the said capillary tubes of claim 13 to excite fluorescence from sample streams.

16. The photon detector for the sheath flow cuvette array of claim 13 is preferably a CCD, positioned at one wide side of the cuvette array of claim 13 and behind a spectral filter to detect the fluorescence from sample streams imaged by a camera lens positioned in front of the said CCD.
